(19) United States
(12) Patent Application Publication
(10) Pub. No.: US 2009/0276965 A1
(43) Pub. Date: Nov. 12, 2009

(54) Glyoxal-alkyl polyglycol ether acetals

(76) Inventor: Frank-Peter Lang, Hattersheim (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/919,650

(22) PCT Filed: May 6, 2006
(86) PCT No.: PCT/EP2006/004273

§ 371 Date: Dec. 1, 2008

(30) Foreign Application Priority Data
May 10, 2005 (DE) 10 2005 021 444.4

(51) Int. Cl. C07C 43/303; D06B 1/00; B08B 3/08

(52) U.S. Cl. 8/139; 568/603; 134/42

(57) ABSTRACT

The invention relates to amphiphilic acetal of the formula (1)

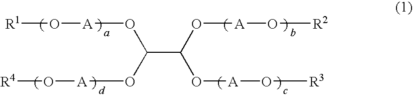

wherein R¹ is n-C₈-C₂₂-alkyl, iso-C₉-C₂₂-alkyl, n-C₅-C₁₈-alkylphenyl or iso-C₅-C₁₈-alkylphenyl, R² and R⁴ are each independently C₁-C₇-alkyl or have the same meaning as R¹, R³ is C₁-C₇-alkyl, A is a group of the formulae —C₂H₄— or —C₃H₆—, and the indices a, b, c and d are each independently from 0 to 50, where at least one of the indices a, b, c or d has to have a value other than 0. The compounds of the invention are suitable as surfactants, emulsifiers, demulsifiers, humectants, foamers, or antifoaming agents.

Tetramethoxyethane, a bisacetal, which is obtained by reacting glyoxal with methanol, has very interesting dissolution behavior. It has comparable dissolution behavior to, for example, ethanol or isopropanol in any mixing ratio with water. At the same time, however, it has distinctly greater dissolution capacity for fats and oils. For example, greasy and oily stains on textiles are sometimes detached equally well as with the nonpolar solvents perchloroethylene or with isoalkanes (“washbenzine”). For this reason, tetramethoxyethane appears to be suitable for use in laundry detergents and cleaning compositions for textiles (for example in spot removers or washing enhancers) or for hard surfaces, for example as a replacement for other organic solvents such as monohydric alcohols (ethanol, isopropanol) dihydric or polyhydric alcohols, (1,2-propylene glycol, glycerol), ethers, in particular glycol ethers, ketones, esters, oligo- and polyalkylene glycols (PEG 300 and PEG 400), terpenes, n- and isoalkanes and nitrogen-containing solvents, for example N-methylpyrrolidone.

Such solvents are used, for example, in liquid laundry detergents, prewash sprays, spot removers, all-purpose cleaners, glass cleaners or specialty cleaners, for example in the automotive sector.

It is also conceivable to use tetramethoxyethane as a substitute for perchloroethylene “PER” or for hydrocarbon solvents “HCS” in dry cleaning.

In spite of its good dissolution capacity for fats and oils, even better dissolution capacity for nonpolar substances and thus even more nonpolar character of the solvent may be required in specific cases.

This becomes possible by exchange of the methyl groups of the tetramethoxyethane for longer alkyl radicals by reacting glyoxal with longer-chain alcohols, for example ethanol, propanol or butanol, and thus obtaining tetraethoxyeethane, tetrapropoxyethane or tetrabutoxyethane. Such symmetrical bisacetals of glyoxal with short-chain linear alcohols having from 2 to 6 carbon atoms and with isooctanol are known. However, the disadvantage of these symmetrical bisacetals is the water solubility which declines with increasing carbon chain length.

It is thus an object of the present invention to provide bisacetals of glyoxal which have even better dissolution capacity for fats and oils and for nonpolar organic solvents than the known glyoxal acetals without simultaneously experiencing a distinct lowering of their water solubility.

It has now been found that, surprisingly, compounds of the formula (1) specified below have amphiphilic properties which are comparable to those of customary surfactants.

The invention provides amphiphilic acetals of the formula (1)

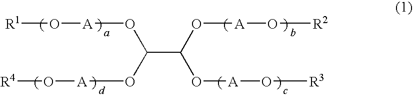

in which R¹ is n-C₈-C₂₂-alkyl or iso-C₉-C₂₂-alkyl or n- or iso-C₅-C₁₈-alkylphenyl, R² and R⁴ are each independently C₁-C₇-alkyl or as defined for R¹, R³ is C₁-C₇-alkyl, A is a group of the formulae —C₂H₄— or —C₃H₆—, and the indices a, b, c and d are each independently from 0 to 50, where at least one of the indices a, b, c or d has to have a value unequal to 0.

In the definition of R¹, preference is given to n-C₁₀-C₁₈-alkyl, n-C₁₂-C₁₆-alkyl, iso-C₁₁-C₁₅-alkyl, iso-C₁₃-C₁₅-alkyl, and also to n- and iso-C₈-C₁₂-alkylphenyl. Preferred C₁-C₇-alkyl (R³) is methyl, ethyl, n-propyl, isopropyl, n-butyl, isobutyl and tert-butyl. A is preferably —C₂H₄—.

The indices a, b, c and d are preferably from 1 to 25, more preferably from 2 to 10 and most preferably from 3 to 8.

Of the compounds of the formula 1, preference is given to the following groups of compounds:

Compounds of the formula 1 in which R¹ is n-C₈-C₂₂-alkyl, preferably n-C₁₀-C₁₈-alkyl, in particular n-C₁₂-C₁₆-alkyl or iso-C₉-C₂₂-alkyl, preferably iso-C₁₁-C₁₅-alkyl, in particular iso-C₁₃-C₁₅-alkyl, and R², R³ and R⁴ are each C₁-C₇-alkyl, A is —C₂H₄—, a is from 1 to 25, preferably from 2 to 10 and more preferably from 3 to 8, b, c and d are each from 0 to 25, preferably from 0 to 3 and more preferably 0.

Compounds of the formula 1 in which R¹ and R² are each n-C₈-C₂₂-alkyl, preferably n-C₁₀-C₁₈-alkyl, in particular n-C₁₂-C₁₆-alkyl or iso-C₉-C₂₂-alkyl, preferably iso-C₁₁-C₁₅-alkyl, in particular iso-C₁₃-C₁₅-alkyl, and R³ and R⁴ are each C₁-C₇-alkyl, A is —C₂H₄—, a and b are each from 1 to 25, preferably from 2 to 10 and more preferably from 3 to 8, c and d are each from 0 to 25, preferably from 0 to 3 and more preferably 0.

Compounds of the formula 1 in which R¹ and R⁴ are each n-C₈-C₂₂-alkyl, preferably n-C₁₀-C₁₈-alkyl, in particular n-C₁₂-C₁₆-alkyl or iso-C₉-C₂₂-alkyl, preferably iso-C₁₁-C₁₅-alkyl, in particular iso-C₁₃-C₁₅-alkyl, and R² and R³ are each C₁-C₇-alkyl, A is —C₂H₄—, a and d are each from 1 to 25, preferably from 2 to 10 and more preferably from 3 to 8, b and c are each from 0 to 25, preferably from 0 to 3 and more preferably 0.

Compounds of the formula 1 in which R¹ is n-C₈-C₂₂-alkyl, preferably n-C₁₀-C₁₈-alkyl, in particular n-C₁₂-C₁₆-alkyl or iso-C₉-C₂₂-alkyl, preferably iso-C₁₁-C₁₅-alkyl, in particular iso-C₁₃-C₁₅-alkyl, and R², R³ and R⁴ are each C₁-C₇-alkyl, A is —C₂H₄—, a is 0, and b, c and d are each from 1 to 25, preferably from 2 to 10 and more preferably from 3 to 8.

Compounds of the formula 1 in which R¹ and R² are each n-C₈-C₂₂-alkyl, preferably n-C₁₀-C₁₈-alkyl, in particular n-C₁₂-C₁₆-alkyl or iso-C₉-C₂₂-alkyl, preferably iso-C₁₁-C₁₅-alkyl, in particular iso-C₁₃-C₁₅-alkyl, and R³ and R⁴ are each C₁-C₇-alkyl, A is —C₂H₄—, a and b are 0, and c and d are each from 1 to 25, preferably from 2 to 10 and more preferably from 3 to 8.

Compounds of the formula 1 in which R¹ and R⁴ are each n-C₈-C₂₂-alkyl, preferably n-C₁₀-C₁₈-alkyl, in particular n-C₁₂-C₁₆-alkyl or iso-C₉-C₂₂-alkyl, preferably iso-C₁₁-C₁₅-alkyl, in particular iso-C₁₃-C₁₅-alkyl, and R² and R³ are each C₁-C₇-alkyl, A is —C₂H₄—, a and d are 0, and b and c are each from 1 to 25, preferably from 2 to 10 and more preferably from 3 to 8.

The compounds of the formula 1 can be prepared by methods of acetalization known per se from glyoxal and alcohol alkoxylates or alkylphenol alkoxylates of the formulae

R¹—(O-A)ₐ-OH,

R²—(O-A)_{b}-OH,

R³—(O-A)_{c}-OH,

R⁴—(O-A)_{d}-OH.

In the case that A=-C₂H₄—, the compounds are the particularly preferred alcohol ethoxylates or alkylphenol ethoxylates.

When a, b, c or d are equal to 0, the compounds are the nonalkoxylated alcohols which can be used to acetalize the glyoxal in a mixture with the alcohol alkoxylates or alkylphenol alkoxylates.

Useful alcohol alkoxylates include, for example, the ethoxylates of native fatty alcohols such as coconut fatty alcohol, lauryl alcohol, myristyl alcohol, oleyl alcohol, tallow fatty alcohol and stearyl alcohol or else the ethoxylates of synthetic alcohols of the oxo alcohol type, of the Ziegler alcohol type or of the Guerbet alcohol type. Useful alkylphenol ethoxylates include, for example, nonylphenol ethoxylate, isooctylphenol ethoxylate and tributylphenol ethoxylates.

The acetalization is effected preferably in the presence of a small amount of an acidic catalyst.

Useful acidic catalysts include both Lewis and Brønsted acids, for example zirconium sulfate, sulfuric acid, methylsulfonic acid, p-toluenesulfonic acid, trichloroacetic acid, oxalic acid or acidic ion exchangers.

The glyoxal used is preferably in the form of an aqueous solution, the aqueous solutions used appropriately being the customary industrial aqueous solutions having a glyoxal content of from 20 to 60% by weight, preferably from 30 to 50% by weight. It is also possible to use crystalline glyoxal, as the trimer having two mols of water of crystallization, for the acetalization.

Depending on the type of the alcohol alkoxylate or of the alkylphenol alkoxylate, it may be advantageous to carry out the acetalization in an inert aprotic organic solvent. In order to shift the reaction equilibrium in the acetalization direction, the water of reaction which occurs is distilled off azeotropically.

The compounds of the formula 1 can be prepared, for example, by acetalizing glyoxal with a mixture of a relatively long-chain alcohol ethoxylate or an alkylphenol ethoxylate and a short-chain alcohol such as methanol, ethanol, propanol or butanol. Depending on whether the glyoxal bisacetal formed is to contain one, two or three relatively long-chain alkyl ethoxylate or alkylphenyl ethoxylate radicals, this and the short-chain alcohol are used in the molar ratios of 1:3 or 1:1 or 3:1.

A further means of preparing compounds of the formula 1 consists in the reaction of glyoxal with a mixture of a long-chain alcohol or an alkylphenol and glycol ethers, for example mono-, di- or tripropylene glycol monomethyl ether, mono-, di- or triethylene glycol mono-n-butyl ether.

Here too, depending on whether the glyoxal bisacetal formed is to contain one, two or three relatively long-chain alkyl or alkylphenyl radicals, the long-chain alcohol or the alkylphenol and the glycol ether are used in the molar ratios of 1:3 or 1:1 or 3:1.

The inventive acetalization of glyoxal can be carried out with an excess of the hydroxyl containing components R¹—(O-A)ₐ-OH, R²—(O-A)_{b}-OH, R³—(O-A)_{c}-OH or R⁴—(O-A)_{d}-OH.

Compounds of the formula 1 in which R¹ and R⁴ are each a relatively long-chain alkyl radical and R² and R³ are each C₁-C₇-alkyl can also be prepared by first fully acetalizing glyoxal with a single alcohol alkoxylate or an alcohol. The thus obtained bisacetal is then reacted again with glyoxal according to EP-0 847 976 to obtain the glyoxal monoacetal. This in turn is then reacted again with another alcohol alkoxylate or another alcohol to give the unsymmetrical glyoxal bisacetal.

A further means of preparing compounds of the formula 1 consists in transacetalization of a glyoxal bisacetal with alcohol alkoxylates. For example, starting from tetramethoxyethane, the bisacetal of glyoxal with methanol, by reacting with from 1 to 3 mol of a fatty alcohol ethoxylate in the presence of acidic catalysts and by distilling off the methanol released, the corresponding mono-, di- or tri(fatty alkyl) glycol ether derivative can be obtained.

The thus obtained compounds of the formula 1 have good dissolution capacity for hydrophobic substances, for example for soil or greases or oils. In addition, they have distinct surfactant behavior, i.e. a reduction in the surface tension or interface tension, an improvement in the wetting action, adsorption on interfaces, the capacity to form micells, solubilization capacity, emulsification capacity, the ability to form microemulsions, dispersion capacity, foaming capacity, the ability to form liquid-crystalline phases.

Some representative examples of amphiphilic acetals of the formula 1 are:

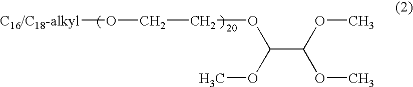

Formula (2): 1,1,2-trimethoxy-2-(C_{16/18}-alkyl polyethylene glycol ether (20 EO))ethane

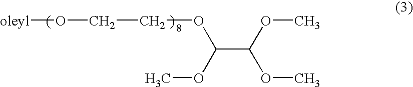

Formula (3): 1,1,2-trimethoxy-2-(oleyl polyethylene glycol ether (8 EO))ethane

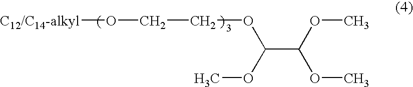

Formula (4): 1,1,2-trimethoxy-2-(C_{12/14}-alkyl triethylene glycol ether)ethane

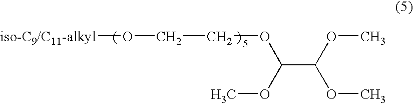

Formula (5): 1,1,2-trimethoxy-2-(iso-C_{9/11}-alkyl pentaethylene glycol ether)ethane

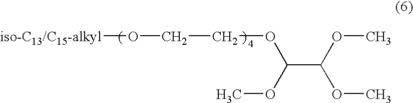

Formula (6): 1,1,2-trimethoxy-2-(iso-C_{13/15}-alkyl tetraethylene glycol ether)ethane

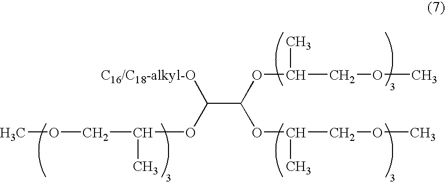

Formula (7): 1,1,2-tri(methylene tripropylene glycol ether)-2-C_{16/18}-alkoxyethane

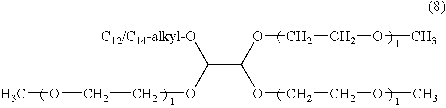

Formula (8): 1,1,2-tri(methyl monoethylene glycol ether)-2-C_{12/14}-alkoxyethane

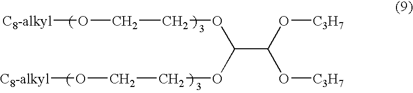

Formula (9): 1,1-di(octyl triethylene glycol ether)-2,2-dipropoxyethane

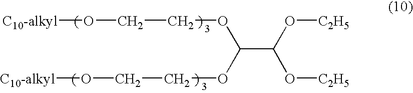

Formula (10): 1,1-di(decyl triethylene glycol ether)-2,2-diethoxyethane

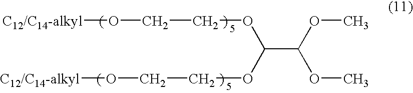

Formula (11): 1,1-di(C_{12/14}-alkyl pentaethylene glycol ether)-2,2-dimethoxyethane

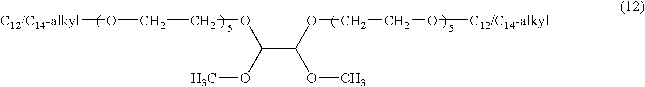

Formula (12): 1-methoxy-1-(C_{12/14}-alkyl pentaethylene glycol ether)-2-methoxy-2-(C_{12/14}-alkyl pentaethylene glycol ether)ethane

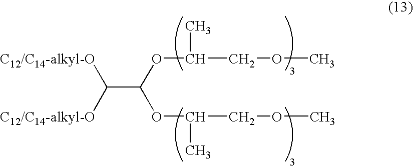

Formula (13): 1,1-di-C_{12/14}-alkoxy-2,2-di(methyl tripropylene glycol ether)ethane

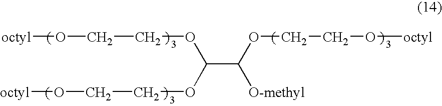

Formula (14): 1,1,2-tri(octyl triethylene glycol ether)-2-methoxyethane

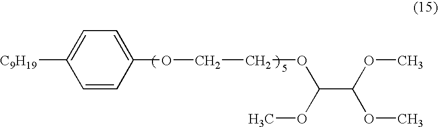

Formula (15): 1,1,2-trimethoxy-2-(nonylphenol polyethylene glycol ether (5 EO))ethane

Depending on the process for the preparation, the desired acetals are not obtained in pure form, but rather as mixtures of different acetals.

The above-described amphiphilic acetals can be used in the following fields: as surfactants, emulsifiers, demulsifiers, dispersants, wetting agents, foamers and defoamers; as laundry detergents and cleaning compositions in aqueous liquor for textiles and for hard surfaces made of coated or uncoated metal, stainless steel, glass, plastics, linoleum, ceramic, porcelain, stoneware, earthenware, wood, concrete, plaster, fired clay or other clay; in dry cleaning as base detergents or in the form of detaching agents or cleaning enhancers; in the textile industry as leveling agents and agents for through-dyeing in the dyeing of textiles, as an emulsifier, as a wetting agent, as a constituent of sizes and as a lubricant, for example in spinning or weaving; as finishes for textiles for changing the surface properties, for example with regard to hand, softness, smoothness, hydrophilicity and hydrophobicity; as antistats for synthetic fibers and plastics; as a constituent of lubricants; as a constituent of metal processing agents, for example in drilling, rolling and cutting oils; in crop protection compositions as emulsifiers, wetters and adjuvants for insecticides, fungicides, herbicides; in leather and fur finishing as wetting agents and detergents or in a mixture with oils as fatliquoring agents; as an additive in the production of paper and also in paper recycling, for example for deinking; as an emulsifier in emulsion polymerization of, for example, polyvinyl chloride, styrene-butadiene rubber (SBR), acrylonitrile-butadiene-styrene (ABS); as a suspension stabilizer in suspension polymerization; in pigment preparations as assistants, for example in the grinding or the dispersion of inorganic and organic color pigments; as additives in paints and coatings, for example as solubilizers, for better wetting of the surfaces to be coated, for rheological modification of the formulations, as leveling agents, and as leveling assistants; in cosmetics, for example as an emulsifier for producing w/o and o/w emulsions; as additives in flotation for extracting ores, in building materials such as cement, concrete, gypsum, for example as superplasticizers or as air-entraining agents; for producing asphalt emulsions, for example as adhesion promoters; as additives in the primary and secondary production of petroleum, for example as emulsion breakers/demulsifiers for removing water from crude oil or as an additive to flowing water to increase the crude oil yield; and as fuel additives.

1. An amphiphilic acetal of the formula (1) 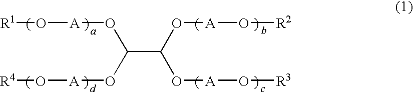
in which R¹ is n-C₈-C₂₂-alkyl or iso-C₉-C₂₂-alkyl or n-C₅-C₁₈-alkylphenyl or iso-C₅-C₁₈-alkylphenyl,
R² and R⁴ are each independently C₁-C₇-alkyl or as defined for R¹,
R³ is C₁-C₇-alkyl,
A is a group of the formulae —C₂H₄— or —C₃H₆—, and the indices a, b, c and d are each independently from 0 to 50, where at least one of the indices a, b, c or d is a value unequal to 0.

2. A compound of the formula 1 as claimed in claim 1, wherein R¹ is n-C₁₀-C₁₈-alkyl or iso-C₁₁-C₁₅-alkyl, R² and R⁴ are each C₁-C₇-alkyl or as defined for R¹, and R³ is C₁-C₇-alkyl.

3. A compound of the formula 1 as claimed in claim 1, wherein R¹ is n-C₁₂-C₁₆-alkyl or iso-C₁₃-C₁₅-alkyl, R² and R⁴ are each C₁-C₇-alkyl or as defined for R¹, and R³ is C₁-C₇-alkyl.

4. A compound of the formula 1 as claimed in claim 1, wherein R¹ is n-C₈-C₂₂-alkyl or iso-C₉-C₂₂-alkyl, and R², R³ and R⁴ are each C₁-C₇-alkyl.

5. A compound of the formula 1 as claimed in claim 1, wherein R¹ is n-C₁₀-C₁₈-alkyl or iso-C₁₁-C₁₅-alkyl, and R², R³ and R⁴ are each C₁-C₇-alkyl.

6. A compound of the formula 1 as claimed in claim 1, wherein R¹ is n-C₁₂-C₁₆-alkyl or iso-C₁₃-C₁₅-alkyl, and R², R³ and R⁴ are each C₁-C₇-alkyl.

7. A compound of the formula 1 as claimed in claim 1, wherein R¹ and R² are each n-C₈-C₂₂-alkyl or iso-C₉-C₂₂-alkyl and R³ and R⁴ are each C₁-C₇-alkyl.

8. A compound of the formula 1 as claimed in claim 1, wherein R¹ and R² are each n-n-C₁₀-C₁₈-alkyl or iso-C₁₃-C₁₅-alkyl and R³ and R⁴ are each C₁-C₇-alkyl.

9. A compound of the formula 1 as claimed in claim 1 wherein R¹ and R² are each n-C₁₂-C₁₆-alkyl or iso-C₁₃-C₁₅-alkyl and R³ and R⁴ are each C₁-C₇-alkyl.

10. A compound of the formula 1 as claimed in claim 1 wherein R¹ and R⁴ are n-C₈-C₂₂-alkyl or iso-C₉-C₂₂-alkyl and R³ and R⁴ are each C₁-C₇-alkyl.

11. A compound of the formula 1 as claimed in claim 1, wherein R¹ and R⁴ are each n-C₁₀-C₁₈-alkyl or iso-C₁₁-C₁₅-alkyl and R² and R³ are each C₁-C₇-alkyl.

12. A compound of the formula 1 as claimed in claim 1, wherein R¹ and R⁴ are each n-C₁₂-C₁₆-alkyl or iso-C₁₃-C₁₅-alkyl and R² and R³ are each C₁-C₇-alkyl.

13. A compound of the formula 1 as claimed in claim 1, wherein A is —C₂H₄—.

14. A compound of the formula 1 as claimed in claim 1, wherein a is from 1 to 25, and b, c and d are each from 0 to 25.

15. A compound of the formula 1 as claimed in claim 1, wherein a and b are, each from 1 to 25, and c and d are each from 0 to 25.

16. A compound of the formula 1 as claimed in claim 1, wherein a and d are each from 1 to 25, and b and c are each from 0 to 25.

17. A compound of the formula 1 as claimed in claim 1, wherein a is 0, and b, c and d are each from 1 to 25.

18. A compound of the formula 1 as claimed in claim 1, wherein a and b are each 0, and c and d are each from 1 to 25.

19. A compound of the formula 1 as claimed in claim 1, wherein a and d are each 0, and b and c are each from 1 to 25.

20. A product comprising the compound of claim 1, wherein the product is selected from the group consisting of a surfactant, an emulsifier, a demulsifier, a dispersant, a wetting agent, a foamer, a lubricant, a metal processing agent, an adjuvant in crop protection, an assistant in pigment preparations, an additive for ore floatation, an additive in fuels, an additive in the production of petroleum, a cosmetic, an additive in paints and coatings, an emulsifier in emulsion or suspension polymerization, an adhesion promoter in asphalt emulsion, a superplasticizer or air-entraining agent in building materials, a wetting and detergent in fur and leather finishing, a fat liquoring agent in a mixture with oils, and a defoamer.

21. A method for cleaning a textile or a hard surface, said method comprising contacting the textile or the hard surface with a cleaning composition in an aqueous liquor comprising the compound of claim 1.

22. An agent for dry cleaning, said agent comprising the compounds of the formula 1 as claimed in claim 1, said agent for dry cleaning selected from the group consisting of base detergents, detaching agents, and cleaning enhancers.

23. An agent for textile industry, said agent comprising the compound of claim 1, wherein the agent is selected from the group consisting of leveling agents, agents for through-dyeing in the dyeing of textiles, an emulsifier, a wetting agent, a constituent of sizes, a finishing agent, an antistat agent, and a lubricant in spinning or weaving.

24-41. (canceled)
